(19) United States
(12) Patent Application Publication
(10) Pub. No.: US 2004/0019300 A1
(43) Pub. Date: Jan. 29, 2004

(54) Microfluidic blood sample separations

(76) Inventor: Leslie Anne Leonard, Portola Valley, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/205,796

(22) Filed: Jul. 26, 2002

(51) Int. Cl. A61B 5/00

(52) U.S. Cl. 600/584

(57) ABSTRACT

A microfluidic system isolates blood cells of a minute volume of a blood sample by using a passive-settlement approach or an active-flow approach, rather than an approach that requires centrifugation. In the passive-settlement approach, the microfluidic system yields a hematocrit measurement by detecting the volume of red blood cells that settle under the influence of gravitational force. According to the active-flow approach, blood cells are isolated at one or more porous regions having pores dimensioned which preferentially pass constituents of the blood sample.

### TECHNICAL FIELD

The invention relates generally to isolating blood cells of a blood sample and more particularly to determining measurements for a small volume of blood.

### BACKGROUND ART

In clinical chemistry, the identification of the composition of a person's blood is used as an important diagnostic tool. Blood is primarily serum, but includes three major types of cells. Serum comprises approximately sixty percent to seventy percent of a human blood sample, while approximately thirty to forty percent of the sample is cellular. Plasma within the sample is more than ninety percent water, with the remainder consisting of proteins, lipids, salts and the like. The three major blood cell types are red blood cells (RBCs), white blood cells (WBCs) and platelets.

The category “hematology” encompasses a wide variety of different measurements, including hemoglobin, hematocrit, total RBC count, total WBC count, total platelet count, differential WBC count, platelet function and calculated RBC indices. Additionally, newer tests are becoming standard additions to this list, including RBC morphology, reticulocyte counts, and neutrophil maturation. Routinely, a large quantity of blood is drawn from a vein in the arm of a person when clinical lab tests are requested. Typically, more than ten milliliters (mis) are drawn. This sample volume is significantly more than is needed for one test, but it is common to run multiple clinical tests.

For tests that do not require cell-related counts, small scale filtration devices are available for at least significantly reducing the hematocrit content. That is, such devices may be used to yield plasma having little or no hematocrit content. U.S. Pat. No. 6,319,719 to Bhullar et al. describes a capillary hematocrit separation structure. A capillary pathway from an inlet port is dimensioned so that the driving force for the movement of a blood sample through the pathway is capillary pressure. Obstacles are fixed within the capillary pathway. Each obstacle has a concave portion facing a reaction region at the downstream side of the obstacles. The obstacles can take on a variety of shapes, but should be dimensioned so that the hematocrit accumulates within the concave portions.

In the case of blood sample analysis that requires cellular considerations, available analyzers range from small benchtop instruments with limited menus to large automated analyzers with significant versatility. The minimum required volume of blood also varies considerably. For example, a large state-of-the-art analyzer may require 150 microliters (μl) of whole blood for a full five-part differential, while a more limited analyzer may require a volume of 12 μl whole blood for a menu of five measured parameters.

As previously noted, one procedure for assessing the health of a patient is to determine the patient's hematocrit. The hematocrit is the percentage of the volume of a blood sample that is occupied by red blood cells. This measurement may be acquired using different techniques. Most comprehensive hematology analyzers derive the hematocrit value from several other direct and indirect measurements. First, the RBC count and a quantitative hemoglobin measurement are obtained. Then, the RBC index, “mean cell hemoglobin content” (MCHC), is calculated for the sum of the RBC count and the hemoglobin measurement. This parameter is then used to calculate hematocrit. Typically, at least one of the measurements is acquired using centrifugal separation of cell populations.

Microhematocrit techniques commonly employ centrifugal separation. One such technique is to use a small capillary tube which is filled with anticoagulated blood and centrifuged under selected conditions, e.g., for five minutes at 14,500 revolutions per minute. Because of the higher density of the red blood cells, these cells collect at the bottom of the tube. Thus, the volume of packed RBCs relative to the total volume of whole blood can be expressed as the percent hematocrit. This is often a relatively inexact measurement, giving values with +/−five percent variation. However, the technique is inexpensive and is used widely. U.S. Pat. No. 6,204,066 to Wardlaw describes a method of determining the sedimentation rate of erythrocyte. An anticoagulated sample of whole blood is placed in a transparent capillary tube, which is then subjected to centrifugation. The position of the erythrocyte/plasma interface in the blood sample is determined at selected time intervals during the centrifugation of the blood sample. The interface positions can be mapped. A value which reflects the sedimentation rate of the sample can be derived from a calculated slope and a Y intercept of the calculated slope, thereby arriving at a conventional gravity sedimentation rate value.

A modification of this technique is to add a plastic “float” to the blood sample to enhance the separation of cell types. Additionally, chemical and fluorescent coatings on the microtube may be used to stain the individual cell types as they migrate past a position, so as to label discrete layers with different fluorescence or absorption. Again, the red blood cells migrate to the bottom of the tube. The hematocrit is obtained directly from the measurement of the volume of red blood cells relative to the total volume of the blood sample.

U.S. Pat. No. 6,350,613 to Wardlaw describes another approach. In this approach, a whole blood sample is placed within a sample chamber having convergent opposing walls. At least one of the convergent walls of the chamber is transparent, so that the blood sample can be observed. By mixing fluorescent dyes with a blood sample, target cells in the sample can be enumerated and differentiated by using a scanning instrument which is able to measure different wavelength color signals emitted from target cells. The varying thicknesses provided by the convergent walls produce a first thickness region in which red blood cells and quiescent monolayers of red blood layers of the sample will reside after the sample fills the chamber. Larger constituents, such as white blood cells and nucleated red blood cells, will reside in greater thickness regions of the chamber. The non-nucleated red blood cells which reside in the greater thickness regions will agglomerate to form rouleaux. A related invention is described in U.S. Pat. No. 6,235,536 to Wardlaw.

While the prior art techniques operate well for their intended purposes, further improvements are desired. For example, in the determination of a hematocrit measurement, centrifugation is typically required, so that the patient or the blood sample must enter a laboratory setting. Moreover, a relatively large sample volume is often required. What is needed is an approach that yields sufficiently accurate cellular measures from a minute quantity of blood. What is also needed is a more convenient approach to acquiring hematocrit measurements and other measurements that require blood cells to be isolated, thereby enabling Point-of-Care testing.

### SUMMARY OF THE INVENTION

A microfluidic system is used to isolate blood cells from other constituents of a minute volume of a blood sample, without requiring centrifugal separation. According to one approach, the separation is carried out by allowing gravitational force to provide separation on the basis of differences in densities. A density gradient medium having a density less than that of at least one blood cell type (RBC), but greater than that of plasma, may be introduced into a microfluidic separation region to allow the cells present in the minute volume of blood to settle through the density medium. In a second approach, the microfluidic system includes a microfluidic path having at least one porous region with pores that are dimensioned to inhibit passage of a specific blood cell type. In one contemplated embodiment, a first porous region includes pores dimensioned to block passage of white blood cells, which are typically the largest constituents of a blood sample, while a second porous region has pores dimensioned to block passage of red blood cells.

The term “microfluidic” feature, as applied to flow regions, such as inlets, outlets and flow channels, shall be defined herein as a feature having a cross sectional dimension (e.g., width, height or diameter) of no more than 500 μm and preferably no more than 200 μm. As applied to non-flow regions, the term “microfluidic” is used herein to define a region in which fluids are introduced and removed via microfluidic features.

In the gravity-separation approach, the microfluidic system yields a hematocrit measurement by detecting the volume of red blood cells within a known volume of blood. The separation region may be a microfluidic channel or may be a chamber. The density medium ensures at least one well defined gradation that is based upon relative densities of blood constituents. Due to the laminar flow properties inherent in microfluidics, the blood fluid will tend to remain on top of the density medium, resisting any mixing of layers. However, the red blood cells will fall through the density medium and will settle on the lower surface of the microfluidic channel or chamber. The layer of red blood cells can then be detected using any of a number of means, such as optical, electrical, and/or thermal. The volume of red blood cells is compared to the total volume of the blood sample to provide the hematocrit measurement. A density gradient medium may be selected to also allow passage of white blood cells. The portion of white blood cells is small compared to the portion of red cells, so that the white cell “contamination” can be readily factored into the hematocrit measurement.

Typically, when blood is drawn and allowed to stand undisturbed, red blood cells naturally settle by gravity. This is because the red cells are denser than the white blood cells and platelets. The age-dependent buoyant density of the red cells is in the range of 1.085 to 1.12, as compared to 1.077 for white blood cells. Thus, the less dense cell types remain at the interface of the aqueous plasma and the settled red cells. In a 10 milliliter blood collection tube, the settling may take hours. However, using the present invention, the hematocrit measurement may be acquired in a time frame of 1 to 2 minutes. An advantage of the use of density gradient medium over passively allowing the red blood cells to settle from a volume of blood is that the medium provides a “sharper” demarcation of blood constituents, thereby providing a more easily detected interface from which to base hematocrit measurements.

Alternatively, porous structures can be used to provide blood cell isolation. Red blood cells tend to have a mean cross sectional dimension of 7 μm, while white blood cells have a mean cross sectional dimension of approximately 20 μm. The differences in sizes can be used to broadly separate these two types of blood cells from other blood constituents.

In one embodiment of this approach in which blood constituents are separated by size, red blood cells can be concentrated into a discrete volume by using a first porous region to separate white blood cells and a subsequent porous region to separate the red blood cells from the plasma and the platelets. The volume of red blood cells can be compared to the volume of red blood cells plus plasma, giving a hematocrit measurement as a percentage. A correction can be made for the presence of low percentages of white cells in the plasma volume. Either a pressurized flow or gravitational force may be used to direct a blood sample through the porous region. Each porous region may be formed by a single layer having holes or may be formed of a number of different structures that are spaced apart by a minute distance to selectively block types of blood constituents.

One advantage of the invention is that cell separation and hematocrit measurements can be obtained in a microfluidic platform without the requirement of centrifugation. Thus, a point-of-care diagnostic tool can be fabricated. Another advantage of the invention is that smaller blood volumes are required. In the current centrifugation devices, required volumes as low as 10 μl have been used, but the present invention allows hematocrit measurements to be performed using a blood sample of 1 μl.

### BRIEF DESCRIPTION OF THE DRAWINGS

FIG. 1 is a perspective view of one embodiment of a microfluidic system for providing blood sample separation in accordance with the invention.

FIG. 2 is a block diagram of components of the microfluidic system of FIG. 1 connected to a source of a blood sample.

FIG. 3 is a representation of constituents of a blood sample separated using a passive-settlement approach in accordance with the invention.

FIGS. 4 and 5 are side views of a microfluidic channel of FIG. 1 having a single porous region for isolating constituents of a blood sample.

FIG. 6 is a side view of a microfluidic channel of FIG. 1 having two porous regions for isolating constituents of a blood sample.

FIG. 7 is a side view of a microfluidic channel of FIG. 1 having a porous region that is formed in a manner different from the porous region of FIG. 5.

FIG. 8 is a side view of a microfluidic channel of FIG. 1 having two porous regions formed in a manner consistent with that of FIG. 7.

FIG. 9 is a side view of a microfluidic channel of FIG. 1 in which a porous region is positioned along a vertical segment of the microfluidic channel.

### DETAILED DESCRIPTION

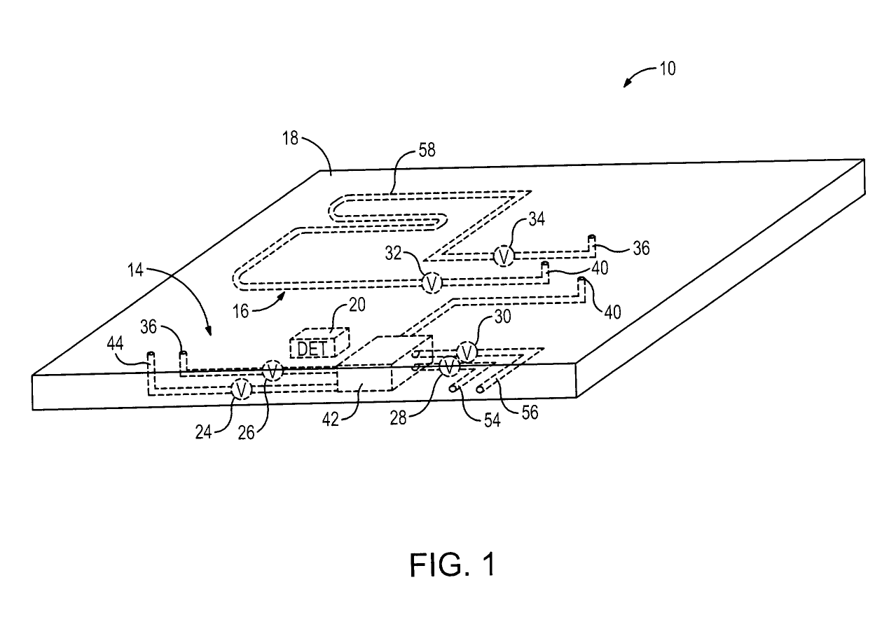

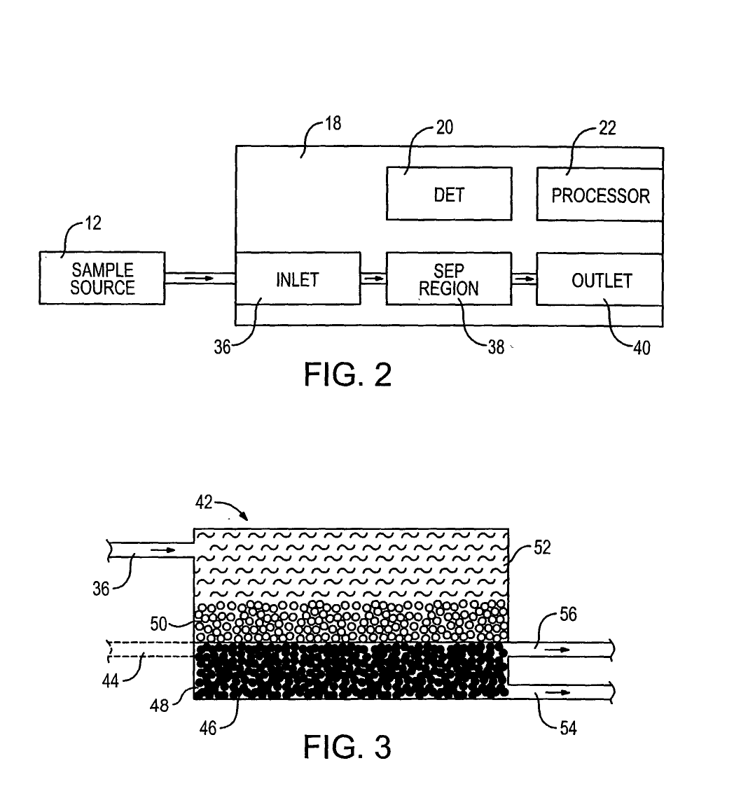

With reference to FIGS. 1 and 2, a microfluidic system 10 may be connected to a source 12 of a blood sample. Two different configurations 14 and 16 of microfluidic features are shown in FIG. 1 for isolating selected constituents of a blood sample, but the system is more likely to use a single configuration. The first configuration utilizes a passive-settlement approach to induce blood cell separation, while the second configuration 16 utilizes an active-flow approach. Both of these approaches will be described in greater detail as follows.

The microfluidic system 10 includes a sampling body 18. The sampling body may be a substrate made of a material such as a polymer, glass, silicon, or ceramic. Polymers are often used in fabricating microfluidic devices. Polymer materials that are well suited for this application include materials selected from the following classes: polyimide, polycarbonate, polystyrene, polyester, polyamide, polyether, polyolefin, and mixtures of such materials.

In the embodiment of FIGS. 1 and 2, the sampling body 18 includes electronic features, mechanical features, and fluidic features. The electrical features include a detector 20 and a processor 22. The electrical features may be integrated into the substrate or may be separately attached. For example, the processor may be an integrated circuit chip that is separately fabricated and then connected to the substrate material. The mechanical features that are represented in FIG. 1 are six valves 24, 26, 28, 30, 32 and 34. However, the valves are not included in embodiments in which blood samples are manipulated through passageways by capillary action. FIG. 2 represents a simplification of the fluidic features, since the drawing only shows an inlet 36, a separation chamber 38, and an outlet 40.

The first configuration 14 of microfluidic features will be described with reference to FIGS. 1 and 3. In this embodiment, flow from the inlet 36 is controlled by capillary action or by the valve 26 to allow a blood sample to enter a separation chamber 42. In a passive device, the microfluidic structure should be designed to enhance blood flow to the bottom of the separation chamber. The inlet flow may be pressurized to ensure that the entire sample volume reaches the chamber. A precisely metered volume may be required in some applications, such as a hematocrit measurement device in which the measure of total volume is based upon an input measurement. A second input 44 to the separation chamber may be used to introduce a density medium, which is sometimes referred to as a density gradient medium. During manufacture, the second input may be used to “load” the device with the density medium. Thus, blood samples would be introduced only when the density medium is present. Alternatively, the density medium may be inserted during the fabrication process and the second input may be deleted. However, the density medium is not critical to all applications.

Red blood cells (erythrocytes) are denser than white blood cells and platelets. The age-dependent buoyant density of red blood cells is in the range of 1.085 to 1.125, while the density of the white blood cells may be 1.077. When whole blood is introduced into the separation chamber 42, layers will be formed, as represented in FIG. 3. Red blood cells 46 will rapidly fall through the density medium 48. On the other hand, white blood cells and platelets 50 will rest within the density medium atop the red blood cells. The uppermost layer is the serum portion 52 of the blood sample.

Due to the laminar flow properties inherent in microfluidics, the blood fluid will tend to remain layered on top of the density gradient fluid. At the lowest level of the separation chamber is the red blood cell layer 46. In this position, the red cells can be detected using the detector 20. As one example, the detector may be an optical device which is able to determine the height of the red blood cell layer. The volume of red blood cells can then be compared to the volume of the whole blood that was introduced to the microfluidic system 10, so as to calculate a hematocrit measurement, as is well known by persons skilled in the art. Since the chamber has a known maximum volume, the volume of the red blood cell layer 46 can be compared to the volume of the chamber to yield a micro-hematocrit calculation. In one application, the starting volume of whole blood is 1 μl.

As an alternative to blood analysis on-board the sampling body 18, the layer 46 of red blood cells may be caused to flow through a first outlet 54 to a station (not shown) for analysis. Similarly, the flow of red blood cells may be directed to an off-board analytical station following a hematocrit determination by the detector 20 and processor 22 of FIG. 2. An outlet valve 28 regulates flow of the red blood cell layer 46 through the first outlet 54. A second outlet 56 resides above the first outlet and may be used to control the flow of other separated constituents, such as the white blood cells within the layer 50. The second outlet valve 30 controls flow through the second outlet 56.

In some applications, the plasma may be of interest, in addition to blood cells. Thus, following separation of a blood sample into constituents, the same or different outlets may be used to independently remove the constituents. As other alternatives, the separated blood cells can be independently analyzed on-board the sampling body 18 or the blood cells can be removed for off-board analysis in order to leave the plasma for on-board measurement or more sophisticated analysis.

As a substitute for the separation chamber, a separation capillary may be used. The separation capillary should be oriented to accommodate gravitational separation of blood constituents. Thus, the capillary may be oriented to be vertical when the sampling body 18 is rested on a table in its intended position.

Referring now to the second configuration 16 of microfluidic features in FIG. 1, one or more porous regions may be formed to separate a whole blood sample into constituents. Microfluidic structures may be used to effect separation of cells from the serum. By inserting structures of a given size and/or given orientation, cells can be preferentially blocked. This technique operates best for separating cells having significantly different sizes. For example, red blood cells having a mean diameter of 7 μm can be relatively easily separated from white blood cells, which have a mean diameter of approximately 20 μm. More precisely fabricated structures are necessary to separate different classes of the same cell type, such as separating populations of white blood cells that differ in size by 2 μm to 3 μm.

In FIG. 1, capillary action or an inlet valve 34 regulates flow from an inlet 36 to a microfluidic channel 58 having at least one porous region. Similarly, capillary action or an outlet valve 32 controls flow from the microfluidic channel 58 to the outlet 40.

While the first configuration 14 that was described with reference to FIG. 3 involves passive settling of blood cells, the second configuration typically involves an active fluid flow. Referring to FIGS. 1, 4 and 5, the porous region of the microfluidic channel 58 may be a single structure 60 having an array of pores 62 that are dimensioned to block the flow of blood cells. Thus, the microfluidic channel will have an upstream portion 64 with blood cells and a downstream portion 66 through which cell-free plasma flows. The porous structure 60 may be fabricated as part of the microfluidic channel structure using known micromachining techniques. Alternatively, the porous structure may be separately fabricated and then inserted into the channel. The pores may be uniform in size, but the susceptibility to clogging may be reduced by providing more than one pore size.

In another embodiment, the microfluidic channel 58 of FIG. 6 has multiple porous structures 68 and 70 within the same porous region. The first structure 68 has an array of pores 72 dimensioned to block white blood cells. The pores 74 of the downstream structure 70 are smaller, since they are designed to block the flow of red blood cells. The pores 72 within the upstream structure 68 may have diameters of approximately 20 μm. As a result, white blood cells will be blocked by this structure. On the other hand, the downstream structure 70 may have pores 74 that prevent passage of constituents having sizes greater than 7 μm, so that red blood cells are blocked. The result is close to that obtained by the prior art techniques of centrifugation.

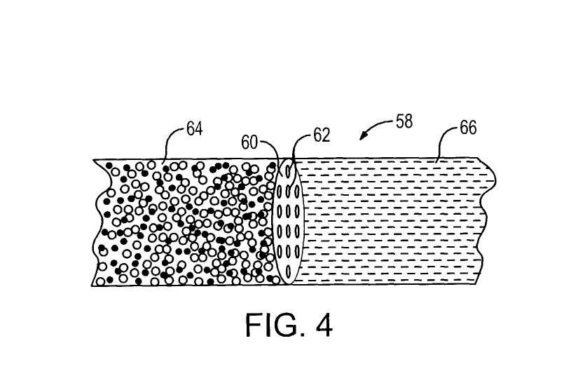

While not shown in FIGS. 4, 5 and 6, the microfluidic channel 58 may be adjacent to a detector, such as an optical detector, which senses the volume of blood cells that are blocked from flowing with the plasma through the porous region. Measurements may then be obtained. For example, a hematocrit measurement may be acquired when the red blood cells are isolated, since the volume of red blood cells can be compared to the total volume of the blood sample. As in the embodiment of FIG. 3, a 1 μl blood sample may be used in the microfluidic system.

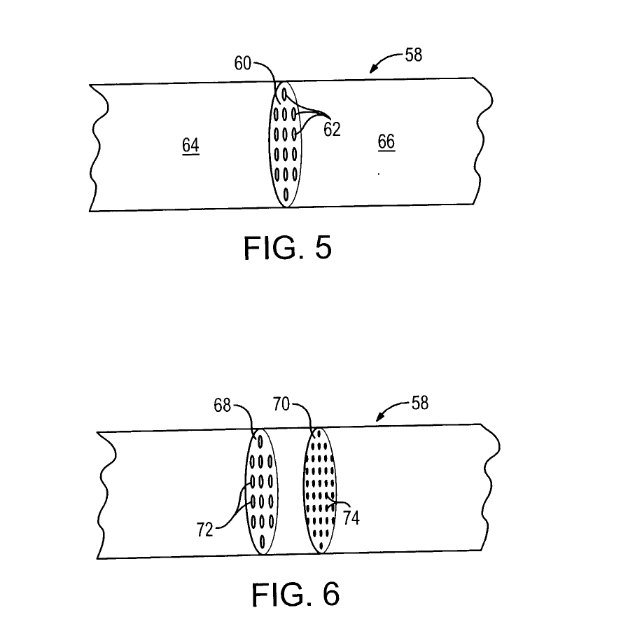

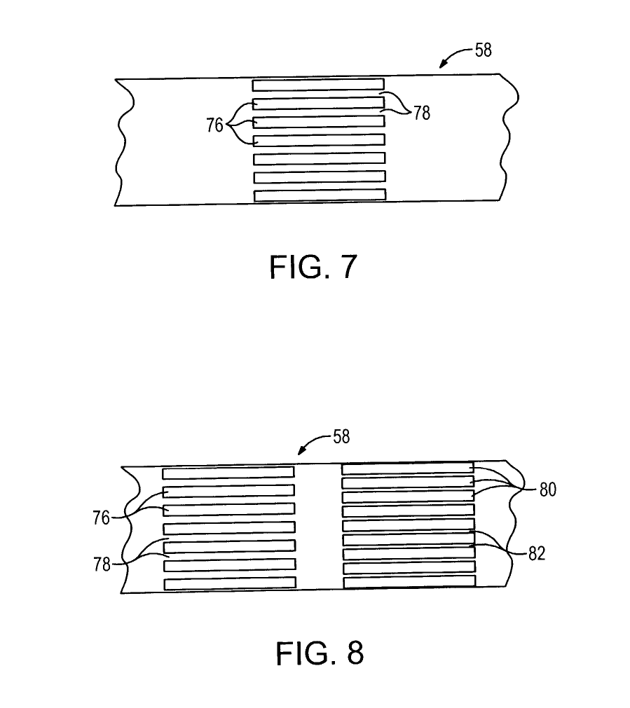

FIG. 7 illustrates another application of the active-flow approach as compared to the passive-settlement approach described with reference to FIG. 3. In FIG. 7, the microfluidic channel 58 includes an array of channel structures 76 which combine to form pores 78 between adjacent structures. As in FIG. 5, the size of the pores is designed to preferentially block constituents of a blood sample. The structure 76 may be layers that are separately fabricated using integrated circuit fabrication techniques. If the spacing between the layers is approximately 20 μm, white blood cells will be blocked, but plasma, platelets and red blood cells will pass through the pores 78. Referring now to FIG. 8, a second array of structures 80 may be positioned downstream of the first array to preferentially block red blood cells. The pores 82 between adjacent structures 80 may be 7 μm. As a consequence, a layer of red blood cells will accumulate between the two arrays of structures. By detecting the volume of the red blood cells between the arrays, a hematocrit measurement may be acquired.

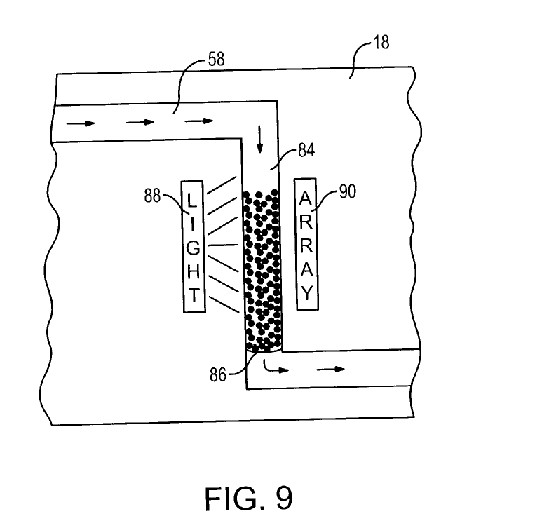

Another embodiment of the active-flow approach is shown in FIG. 9. The microfluidic channel 58 includes a vertical section 84 that operates as the separation region for red blood cells. A porous structure 86 blocks the passage of the red blood cells, but allows the plasma to continue to flow. In the fabrication of the sampling body 18, care is taken to enable light to propagate from a light source 88 to a photosensor array 90. The photosensor array includes at least one column of photosensor elements that individually generate electrical signals in response to light. Thus, when a column of red blood cells is contained within the vertical section 84 of the microfluidic channel 58, light will be blocked from reaching a lower portion of the array 90. As the red blood cells continue to accumulate, a larger portion of the photosensor elements will be blocked. Therefore, the output of the photosensor array may be used as an indicator of the volume of red blood cells within a blood sample.

The embodiments that have been described allow hematocrit measurements to be obtained using a hand-held cartridge. Consequently, point-of-care measurements can be acquired, rather than requiring a patient to travel to a laboratory setting having centrifugation capability or requiring the transportation of a blood sample to such a laboratory setting. Moreover, the minimum required volume for the blood sample is significantly less than the required amount using prior art techniques. A simplified test may be performed by healthcare professionals to evaluate such concerns as infections, anemia, and blood loss.

What is claimed is:

1. A microfluidic system for enabling hematocrit measurements comprising:
a sampling body having microfluidic features formed therein, said features including:
(a) a microfluidic inlet configured to receive a blood sample from a source, said microfluidic inlet extending into said sampling body; and
(b) a separation region connected to said microfluidic inlet and configured to promote separation of red blood cells from plasma of said blood sample, said separation region being cooperative with said microfluidic inlet such that a precisely metered volume of said blood sample is introduced to said separation region; and
a detector enabled to determine a hematocrit measurement for said blood sample on a basis of said separation of said red blood cells.

2. The microfluidic system of claim 1 wherein said separation region is a chamber for retaining said blood sample in position while said red blood cells migrate downwardly relative to other constituents of said blood sample, said downward migration being a result of gravitational force.

3. The microfluidic system of claim 2 further comprising a density medium within said chamber, said density medium having a density less than said red blood cells but not exceeding densities of said other constituents.

4. The microfluidic system of claim 1 wherein said microfluidic features of said sampling body include a cell outlet path from said separation region, said cell outlet path being positioned to preferentially remove said red blood cells following said separation.

5. The microfluidic system of claim 4 wherein said microfluidic features further include an outlet valve coupled to said cell outlet path to selectively enable flow from said separation region via said cell outlet path.

6. The microfluidic system of claim 5 wherein said microfluidic features further include an inlet valve coupled to said microfluidic inlet to selectively enable flow to said separation region.

7. The microfluidic system of claim 1 wherein said microfluidic features of said sampling body further include a porous structure along said separation region, said porous structure being configured to block said red blood cells while permitting passage of other constituents of said blood sample.

8. The microfluidic system of claim 7 wherein said porous structure is a polymer member having pores of a size selected to block said red blood cells.

9. The microfluidic system of claim 8 wherein said microfluidic features further include a second porous structure having pores of a size selected to block white blood cells.

10. A method of isolating cells of a blood sample comprising:
providing a microfluidic system having an inlet and a microfluidic path that includes at least one porous region having pores that are dimensioned to inhibit passage of a specific blood cell type;
directing said blood sample to said at least one porous region;
separating constituents of said blood sample by preferentially passing said constituents through said at least one porous region; and
determining a cellular measure that is specific to said blood sample on a basis of said separating.

11. The method of claim 10 wherein directing said blood sample includes pressurizing said blood sample to provide a continuous flow through said microfluidic path.

12. The method of claim 11 wherein said directing includes positioning said at least one porous region such that said gravitational force causes said constituents of said blood sample to be preferentially passed through said at least one porous region.

13. The method of claim 10 wherein providing said microfluidic system includes providing a first said porous region having pores dimensioned to block white blood cells.

14. The method of claim 13 wherein providing said microfluidic system includes providing a second said porous region having pores dimensioned to block red blood cells.

15. The method of claim 10 wherein said determining includes using said separation of said constituents as a basis for determining a hematocrit measurement.

16. The method of claim 15 wherein providing said microfluidic system includes fixing a detector in a position to detect blood cells blocked by one of said porous regions.

17. A method of isolating cells of a blood sample comprising:
providing a microfluidic system having an inlet and a microfluidic path to a separation region;
providing a density medium within said separation region, said density medium having a density that is less than a density of red blood cells but that does not exceed all densities of blood constituents;
directing a blood sample through said microfluidic path to said separation region, thereby enabling separation of at least some of said blood constituents on a basis of relative densities; and
utilizing a detector to measure a volume of at least one said blood constituent while residing within said separation region.

18. A method of acquiring a blood cell measurement for a blood sample comprising:
providing a microfluidic system having an inlet and a microfluidic separation region;
introducing a density medium in said microfluidic separation region, said density medium having a density less than a density of at least one blood cell type to be separated but greater than plasma of said blood sample;
introducing said blood sample into said microfluidic separation region;
allowing constituents of said blood sample to separate as a consequence of differences in densities of said constituents and said density medium; and
determining a blood cell measurement by detecting a volume of said blood cells separated as constituents of said blood sample.

19. The method of claim 18 wherein introducing said density medium includes selecting a fluid having a density greater than white blood cells and less than red blood cells.

20. The method of claim 18 wherein said determining includes detecting said volume of blood cells on the basis of said blood cells passing through said density medium by gravitational force.

21. The method of claim 20 wherein said determining includes calculating a hematocrit measurement from said detecting.
